(19) United States
(12) Patent Application Publication
(10) Pub. No.: US 2026/0007334 A1
(43) Pub. Date: Jan. 8, 2026

(54) SMALL-SCALE ANALYTE SENSORS

(71) Applicant: Laxmi Therapeutic Devices, Inc., Goleta, CA (US)

(72) Inventors: Peter Rule, Goleta, CA (US); William Peter Van Antwerp, Santa Clarita, CA (US)

(21) Appl. No.: 19/258,209

(22) Filed: Jul. 2, 2025

Related U.S. Application Data
(60) Provisional application No. 63/667,325, filed on Jul. 3, 2024.
(60) Provisional application No. 63/667,427, filed on Jul. 3, 2024.

(51) Int. Cl. A61B 5/145; A61B 5/00

(52) U.S. Cl. CPC A61B 5/14532; A61B 5/14503; A61B 5/1451; A61B 5/6833; A61B 2562/125

(57) ABSTRACT

A monitor for determining analyte concentrations in vivo includes a housing configured to adhere to the skin of a subject, a sensor body configured to extend from the housing into a patient's skin, the sensor body being elongate and extending along a longitudinal axis in a length direction, and further having a width and a thickness each measured perpendicular to the length, and an analyte sensing region formed on the sensor body. The analyte sensing region has opposite ends in the length direction, where a maximum length of the analyte sensing region measured between the opposite ends is 80 μm or less.

### CROSS-REFERENCE TO RELATED APPLICATION(S)

The present application claims priority to and the benefit of both U.S. Provisional Patent Application No. 63/667,325, filed Jul. 3, 2024, the contents of which are hereby incorporated by reference in their entirety, and U.S. Provisional Patent Application No. 63/667,427, filed Jul. 3, 2024, the contents of which are hereby incorporated by reference in their entirety.

### BACKGROUND

### Field

The present disclosure relates to medical devices such as analyte sensors, which can be used to measure analytes, such as glucose levels, in the body of a subject. More specifically, the present disclosure is directed to improvements in sensor members associated with such medical devices, and methods for manufacturing such improved sensor members.

### Description of Related Art

Monitoring different analytes in the human body can be used for various diagnostic reasons. In particular, monitoring glucose levels is important for individuals suffering from type 1 or type 2 diabetes. People with type 1 diabetes are unable to produce insulin or produce very little insulin, while people with type 2 diabetes are resistant to the effects of insulin. Insulin is a hormone produced by the pancreas that helps regulate the flow of blood glucose from the bloodstream into the cells in the body where it can be used as a fuel. Without insulin, blood glucose can build up in the blood and lead to various symptoms and complications, including fatigue, frequent infections, cardiovascular disease, nerve damage, kidney damage, eye damage, and other issues. Individuals with type 1 or type 2 diabetes need to monitor their glucose levels in order to avoid these symptoms and complications.

Analyte monitors, and in particular, glucose monitors for the monitoring of glucose levels for the management of diabetes, are constantly being developed and improved. Although there are several platforms for monitoring analytes such as glucose available on the market, there is still a need to improve their precision, wearability, and accessibility to end-users. In addition, there is a desire to provide robust, less painful, less error-prone, and/or generally more effective continuous glucose monitors which may be attached to the patient's body for a more prolonged period of time, as well as glucose monitor features that can be used together with such improved monitor and monitor designs.

In particular, as continuous glucose monitors improve, for example, as life cycles of monitors have proposed to increase in certain applications and as various different ways have been proposed to provide increased accuracy and decreased discomfort for patients who wear and use such monitors, it has become more difficult to develop sensor members for such continuous glucose monitors that satisfy all of these heightened expectations.

### SUMMARY

Many continuous glucose monitors are intended to be worn on a patient's skin for a duration of multiple days or weeks. Most or all commercially available glucose sensors on the market today sense glucose in interstitial fluid (ISF) below the surface of the skin. Such sensing or monitoring therefore typically involves an initial step of inserting a sensing portion of a sensor member of the glucose monitor under the patient's skin. For the most part, this insertion step will involve puncturing the surface of the skin, for example, with a separate needle, for example, on an applicator to provide access for inserting the sensor. Thereafter, the needle or other sharp may be retracted, while the sensor member stays in place under the patient's skin. The rest of the monitor may be adhered to the patient's skin above the sensor member, and may be physically connected to the sensor member, in order to hold the sensor member in place under the skin, to protect the sensor member from environmental and other external conditions, and to provide and house electronics associated with the analyte monitoring, for example, a batter to provide power to the system, a processor and other circuitry to record and/or analyze electrical signals received from the sensor member, and/or a transmitter to transmit data to an offsite location, for example, a cellular phone or server configured to receive and further process the collected data.

With a sensing region of the sensor member of the monitor implanted into the skin, particularly for prolonged periods of time, trauma and other discomfort caused by the implanted portion of the sensor member to the patient may be magnified. There has therefore been a push to find ways to reduce the discomfort caused by sensor members on end users. However, there have been some hurdles which have been difficult to overcome. For example, manufacturing limitations restricted the ability to produce sensor members at smaller sizes, and/or softer or more flexible sensor members which could have caused less discomfort to users. In addition, if the implanted portions of sensor members were too short, increased likelihood of the sensor members being inadvertently withdrawn or otherwise pulled out from its position under the skin, particularly over the increased life spans of continuous glucose monitors and more opportunities for sensor dislodging, deterred further reduction in sensor member sizes.

As a result, most continuous glucose monitors that are commercially available have settled on sensor members that extend about 6 mm to 7 mm below the surface of the skin, with exposed sensing regions formed on the sensor members that are about 180 μm to 400 μm in length or larger. Human skin includes three layers, an outermost epidermis, an intermediate dermis, and a deeper hypodermis, also called the subcutaneous layer. Based on the above size of most commercially available sensors, the sensor members typically position their sensing regions in interstitial fluid within the hypodermis or subcutaneous layer, which is consistent with the 6 mm to 7 mm depth. Specifically, since sensing regions of sensor members are typically at or near the distal end of their respective sensor members to ensure positioning under the skin, the sensing regions of most commercially available monitors are also positioned in the hypodermis. The prior limitations on sensor size and design discussed above, among other limitations, deterred further reduction in size of sensor members previously on the market.

According to embodiments of the invention, sensor members of analyte monitors, particularly continuous glucose monitors, are capable of being manufactured smaller, while also being able to increase accuracy of data collection, as well as improve retention of the portions of the sensor member implanted into the skin and prevent unintended withdrawal or pulling out of the sensor member and its associated sensing regions. In particular, by reducing a size and adjusting a position of the sensing regions, more accurate sensing results can be obtained, while improvements to the shape and direction of implantation will hold the sensor member in the patient's skin and prevent unintended dislodging of the sensor member therefrom. Manufacturing methods according to embodiments of the invention facilitate and make possible manufacturing of such sensor members with reduced size.

According to an embodiment of the invention, a monitor for determining analyte concentrations in vivo includes a housing configured to adhere to the skin of a subject, a sensor body configured to extend from the housing into a patient's skin, the sensor body being elongate and extending along a longitudinal axis in a length direction, and further having a width and a thickness each measured perpendicular to the length, and an analyte sensing region formed on the sensor body. The analyte sensing region has opposite ends in the length direction, where a maximum length of the analyte sensing region measured between the opposite ends is 80 μm or less.

More specifically, the maximum length of the analyte sensing region measured between the opposite ends may be 40 μm or less.

The monitor may be a glucose monitor configured to determine glucose levels of the patient in vivo.

The analyte sensing region may be positioned near a free end of the sensor body.

A width of the sensor body approximate the analyte sensing region may be greater than a width of another portion of the sensor body away from the analyte sensing region.

When the sensor body extends into the patient's skin, the analyte sensing region may be configured to be held in the dermis of the skin of the patient.

The analyte sensing region may include a plurality of electrodes. The plurality of electrodes may include a working electrode. The plurality of electrodes may further include at least one of a counter electrode or a reference electrode.

When the sensor body extends into the patient's skin, a free end of the sensor body may be configured to be held in the dermis of the skin of the patient.

When the sensor body extends into the patient's skin, the sensor body may extend at an acute angle to a surface of the patient's skin.

The housing may include a base having a surface configured to adhere to the skin of the subject and a separate transmitter configured to be assembled to the base after the sensor body is implanted into the patient's skin.

According to another embodiment of the invention, a sensor member for a monitor that is configured to determine analyte concentrations in vivo includes a sensor body configured to extend into a patient's skin, the sensor body being elongate and extending along a longitudinal axis in a length direction, an analyte sensing region that is formed on the sensor body by utilizing at least one microfabrication technique. The analyte sensing region has opposite ends in the length direction, where a maximum length of the analyte sensing region measured between the opposite ends is 80 μm or less.

More specifically, the maximum length of the analyte sensing region measured between the opposite ends may be 40 μm or less.

The monitor may be a glucose monitor configured to determine glucose levels of the patient in vivo.

The sensor body may have a length such that when the sensor body extends into the patient's skin, a free end of the sensor body is configured to be held in the dermis of the skin of the patient.

The at least one microfabrication technique may include photo lithography.

The at least one microfabrication technique may include ultraviolet (UV) etching.

The at least one microfabrication technique may include electrochemical plating.

The sensor body may be formed on a wafer. The at least one microfabrication technique may include layering functional chemistry on the wafer by spinning as material substrates are deposited as liquid.

According to embodiments of the invention, sensor members for analyte sensors can be manufactured consistently at a much smaller scale, and can further be shaped and deployed to increase retention under a patient's skin after implantation. Sensor members according to embodiments of the invention can remove noise during data collection, which can greatly improve accuracy of the data collected, when compared to existing analyte monitors on the market.

### BRIEF DESCRIPTION OF THE DRAWINGS

Further features and advantages of the invention will become apparent from the description of embodiments by means of the accompanying drawings. In the drawings:

FIGS. 1A and 1B schematically show a human body with an analyte monitor including an analyte sensor according to embodiments of the invention, where the analyte monitor is attached at different positions on the body.

FIG. 2 shows a perspective view from above an exemplary analyte monitor including an analyte sensor according to embodiments of the invention.

FIG. 3 shows a perspective view from below the analyte monitor of FIG. 2.

FIG. 4 shows an enlarged perspective view of a portion of a sensor member of an analyte monitor according to an embodiment of the invention.

FIG. 5 shows a cross-sectional view from a side of a portion of a patient's skin with a schematic depiction of a sensor member of an analyte monitor according to an embodiment of the invention, e.g., the analyte monitor shown in FIGS. 2-3, implanted therein.

FIG. 6 shows a further cutaway view of a portion of a patient's skin with a schematic depiction of a sensor member of an analyte monitor according to an embodiment of the invention implanted therein.

FIG. 7 shows a consensus error grid for different competitor devices that are currently on the market.

FIG. 8 shows a graph that illustrates a relationship between sensor size and respective magnitudes of useable signal and noise, as well as signal-to-noise ratio, for different sized sensing regions.

FIG. 9 shows a Clarke error grid for a sensor member according to an embodiment of the invention.

### DETAILED DESCRIPTION

In the following detailed description, only certain embodiments of the subject matter of the present disclosure are described, by way of illustration. As those skilled in the art would recognize, the subject matter of the present disclosure may be embodied in many different forms and should not be construed as being limited to the embodiments set forth herein.

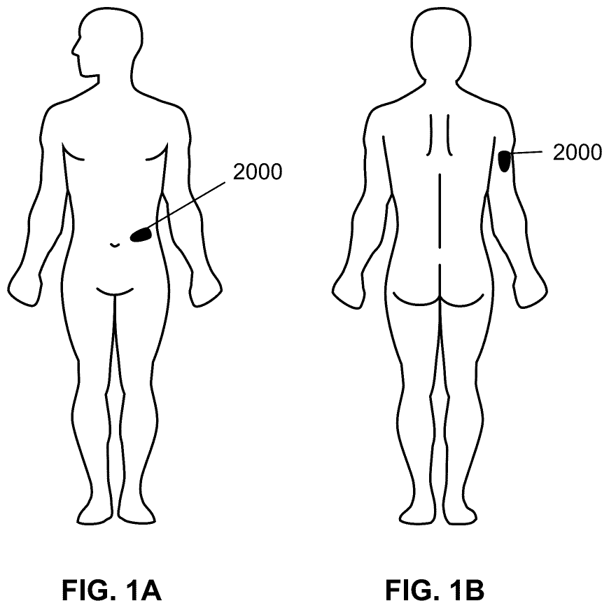

Monitors that include analyte sensors, such as glucose monitors, and in particular continuous glucose monitors, can be attached to a patient's body in different locations, in order to for example, improve glucose monitoring and/or a patient's comfort, since the continuous glucose monitors must remain adhered to the patient's skin, sometimes for a few days or more. FIG. 1A shows a first exemplary analyte monitor 2000 that is adhered to a patient's abdominal region, while FIG. 1B instead shows the exemplary analyte monitor 2000 adhered to a patient's arm. These are only meant to be example adhesion sites, and in other situations, this or a similar analyte monitor may instead be adhered or otherwise attached to other parts of the patient's body.

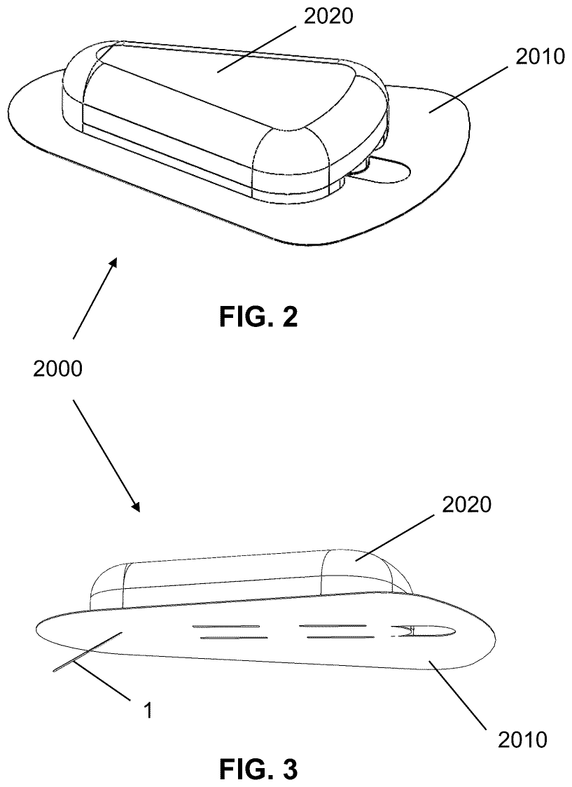

FIGS. 2 and 3 show different schematic views of an example analyte monitor 2000, which can be a continuous glucose monitor, according to an embodiment of the invention. The continuous glucose monitor 2000 may include a base or cradle 2010 that may have an adhesive layer for adhering to a patient's skin, a transmitter 2020 for transmitting data to and/or from a location away from the monitor, and a sensor member 1 which may include an integrated analyte sensing region such as a glucose sensor. It is to be understood that the example analyte monitor shown in FIGS. 2 and 3 are for illustrative and descriptive purposes only, and that analyte monitors with different structures and functionality can also be used in conjunction with the sensor assemblies described below, without departing from the spirit or scope of the invention.

The base 2010 will generally include an adhesive patch or other adhesive mechanism on its lower surface in order to facilitate attachment of the base 2010 to the surface of the patient's skin. In some embodiments of the invention, the sensor member 1 may be integrally formed with the base 2010 so as to be implanted under the patient's skin upon attachment of the base 2010 to the surface of the skin, while in other embodiments the base 2010 may be applied to the skin first, and the sensor member 1 may be advanced to a position where the sensor member 1 becomes attached to the base during or after implantation of part of the sensor member 1 under a patient's skin. For some embodiments, application of the base 2010 and/or the sensor member 1 may be facilitated with one or more applicators.

In some embodiments, after the base 2010 has been attached to a surface of the patient's skin and after at least part of the sensor member 1 has been advanced under the patient's skin, the transmitter 2020 which may be a separate part can be attached to the base. In some embodiments, the transmitter 2020 may include, for example, a power source such as a battery, while the base 2010 and/or the sensor member 1 may include additional electrical circuitry or contacts to complete a circuit, such that the monitor 2000 is powered up upon assembly of the transmitter 2020 to the base 2010. Other embodiments may include other arrangements, for example, where the battery is housed in the base 2010 instead of on the transmitter 2020, etc. Still other embodiments may include further different arrangements, for example, an integrally manufactured monitor where the base and transmitter are formed together in a single main body and which may not be separable from one another by the end user, where such embodiments may also include an integrated sensor member, or may include a sensor member that is implanted through the main body of the monitor after the main body has been adhered to the surface of the patient's skin.

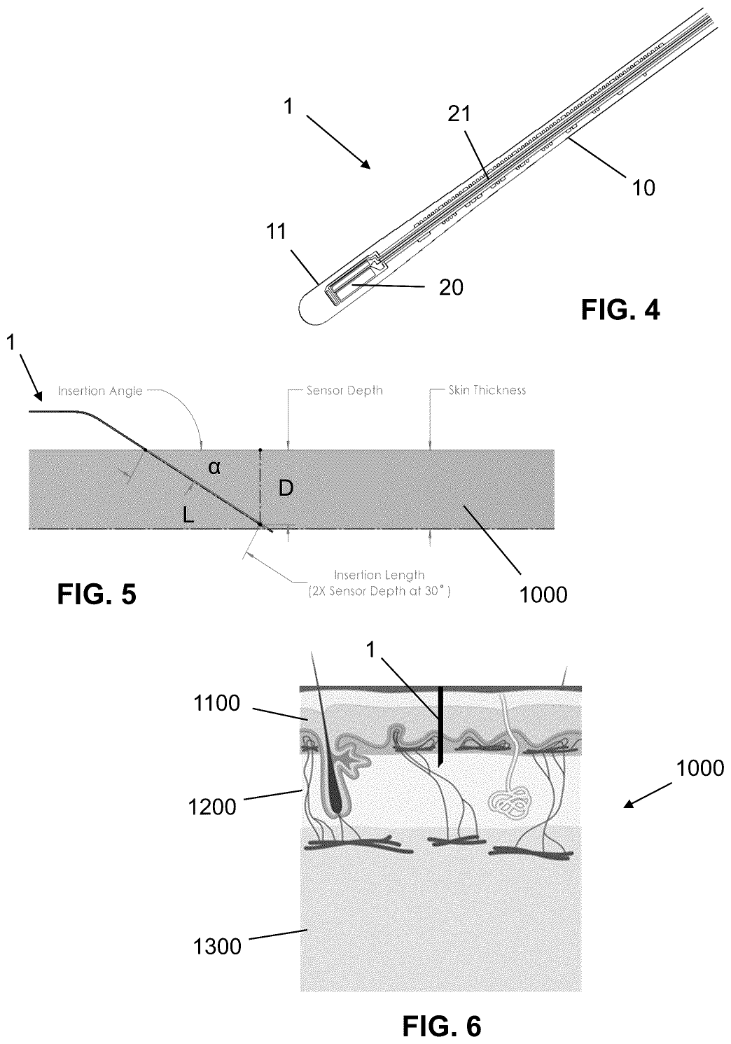

Construction of the sensor member 1 according to an embodiment of the invention will now be discussed in greater detail, with reference to FIG. 4. As seen in FIG. 4, a sensor member 1 may include an implantable portion that includes a main body 10 and a sensing region including one or more sensing electrodes 20. The sensing electrodes 20 may include one or more of a working electrode, a counter electrode, and a reference electrode, where other embodiments may include more or less electrodes, according to the particular application. The main body 10 may include a sensor tip or distal end region 11 that is enlarged in a width direction compared to other portions of the main body. For example, in one embodiment, the distal end region 11 may be substantially teardrop-shaped or otherwise widened with a tapering profile. In other embodiments, the distal end region may include arms or teeth extending laterally outwardly, or may include other shapes or profiles that provide for a varying width. Enlarging the distal end region 11 can help anchor the electrodes at a particular position under the surface of the skin and facilitate improved positioning and retention of the sensor member 1 under the skin after implantation. In one embodiment, the distal end region 11 may be approximately 38% wider than another portion of the main body 10, for example, if a portion of the main body 10 has a width of 300 μm, the distal end region 11 may have a width of 416 μm. Other ratios and sizes may also be contemplated without departing from the spirit or scope of the invention. For example, the distal end region 11 may be 20% to 150% wider than another portion of the main body 10, more preferably 25% to 75% wider than another portion of the main body, and still more preferably 30% to 50% wider than another portion of the main body. The sensing electrodes 20 may further be electrically connected to leads 21 that extend along the main body 10 of the sensor and to an opposite proximal end of the sensor member 1, to facilitate electrical connectivity and communication of the sensing electrodes 20 with other electrical components of the monitor.

According to an embodiment of the invention, the sensor member 1 may further be manufactured or otherwise formed to be more flexible than traditional sensors. Fabrication techniques may be utilized, for example, to fabricate the sensor member 1 according to embodiments of the invention on a thin film substrate or other similar flexible substrate, in order to promote increased flexibility of the sensor. Fabrication techniques can also be utilized to facilitate reducing the thickness of the sensor member 1 according to embodiments of the invention, to further increase flexibility. By way of example, typical stiffer sensor members may be around 200 μm in thickness, whereas sensor members 1 according to embodiments of the invention may be manufactured to be less than half as thick, for example, 75 μm. or even thinner. Other similar reductions in sizes may further be realized without departing from the spirit or scope of the invention.

Sensor members 1 with reduced size according to embodiments of the invention can be manufactured by leveraging microfabrication technologies, such as photo lithography, ultraviolet (UV) etching, and/or electrochemical plating. Functional chemistry can be consistently layered on a wafer, for example, by spinning as material substrates are deposited as liquids, in order to provide for a manufacturing method that yields consistently manufactured sensor members.

While sensing regions of sensor members for existing analyte monitors are typically on the order of 180 μm to 400 μm or more in length, with the improved manufacturing techniques according to embodiments of the invention, sensing regions for sensor members can be manufactured to be as small as 80 μm or less in length, and in some cases as small as 40 μm or less in length, which is significantly smaller/shorter than the sensing regions of sensor members that are currently available. When describing length measurements for sensing regions according to embodiments of the invention, it is to be understood that the measurements are intended to represent a maximum length measurement of the sensing regions. In other words, a length of a particular sensing region as discussed herein may represent a maximum measurement in a length direction between non-sensing regions or non-conductive regions that surround or border the sensing region. Further improvements in fabrication or other manufacturing techniques according to embodiments of the invention may still further yield sensor members with even smaller sensing regions. Reduction in size of sensing regions of sensor members can further have the advantage of allowing for a reduced size of the entire sensor member on which the sensing region is formed as well.

Further, it is to be noted that, while the present disclosure mostly discusses the size of sensing regions according to embodiments of the invention in terms of total length of the sensing regions, reductions in size in other dimensions, for example, in the width direction, is advantageous as well, where reductions in a total area of the sensing region in general will result in improved performance. The respective lengths of embodiments of the invention are only described as a particular reduced dimension, both for simplicity and since length is typically the largest dimension, and it should be understood that embodiments of the invention should not be limited to sensors with length limitations only.

There are many benefits to sensor regions using small area sensing. The use of the smallest feasible electrodes yields advantages for in vivo sensing applications due, for example, to the inhomogeneous nature of tissue and the characteristics of electrochemical systems. Electrochemical sensors perform most optimally in homogeneous matrices, for example, in a homogeneous aqueous solution. Electrodes with small or minimized areas have proven advantageous in biosensing applications because the smaller layout and footprint increases the likelihood that the entirety of the sensor cell, that is, the working electrode, the counter electrode, and/or the reference electrode, as well as any other electrodes intended to be within the vicinity of the working electrode, will come to rest in the same chemical environment within the host tissue, or at least an environment that is more homogenous than for current sensors on the market. On the other hand, since typical sensors have sensing regions with larger intradermal structures, the larger electrode portion or sensing area generally encompasses a larger surrounding environment which may not be homogeneous, or at least less homogeneous than the smaller area or environment encompassed by the smaller sensing regions according to embodiments of the invention.

In particular, reduction in the height or the length direction of sensing regions of sensor members according to embodiments of the invention also facilitates easier locating of the sensing region within a target window in the patient's skin, and allows for more margin for error during positioning. For example, if the dermis of the skin is approximately 1 mm (i.e., 1000 μm) thick, it is easier to locate an 80 μm tall sensing region entirely within the dermis than to locate a larger 400 μm tall sensing region. Additionally, even when properly placed within the dermis, the dermis itself is not entirely homogeneous, and so it is further beneficial to position the entire sensing region within as small a footprint within the dermis as possible, so that the entire sensing region is located within as homogeneous an environment as possible to improve reading accuracy.

To the latter point above, other size reductions in directions other than the length direction, for example, reductions in the widths of the sensing regions, are also beneficial, and can be implemented without departing from the spirit or scope of the invention. For example, just like with height/length considerations, sensing regions with reduced width will also reduce the overall footprint of the sensing region within the dermis, and reduce the potential of part of the sensing region being in an inhomogeneous or less homogeneous region of the surrounding environment within the skin during monitoring. Further modifications are also envisioned, for example, specific shapes of sensing regions that may further promote easier placement of the sensing regions within a more homogeneous environment. For example, some embodiments may include square-shaped sensing regions, circular or other rounded sensing regions, or sensing regions with widths that are greater than their respective lengths so as to be elongate in the width direction, to name just a few examples. In some embodiments, other shapes may also be envisioned, for example, non-uniform or asymmetric shapes may also be realized, and for example, may be tailored to or otherwise chosen based on the anatomy of the surrounding skin of a particular patient, in an effort to further improve accuracy.

The small overall size of the sensing region of the sensor member according to embodiments of the invention also has a benefit in reducing the contribution of solution resistivity in the cell, which may result in lower current offsets and reduced susceptibility to changes in conductivity that may increase measurement noise levels. The uniformity of the measurement environment made possible by reducing the size of the sensing region of sensor members according to embodiments of the invention, coupled with the reduced impact of noise levels and other potential interferences to the measured signal, results in more consistent charge transport and physical diffusion characteristic across the surfaces of the electrodes, which should result in benefits to the stability and accuracy of the sensor member as a whole.

In addition, due to the smaller overall size of the sensor member according to embodiments of the invention, the sensor member reduces the amount of tissue that must be displaced during both insertion and retention. The decreased volume of tissue displaced by the smaller sensor according to embodiments of the invention may also be advantageous in creating a more favorable wound healing response, decreasing pressure on the sensing surface, and/or allowing for more natural circulation of interstitial fluid. These benefits, among others, may further result in reductions in the amount of time required for hydration of the sensor and/or reduced startup time for gathering usable electrochemical measurements.

As noted above, reductions in the size of sensor members have previously been met with difficulties, particularly with respect to properly retaining the smaller sized sensors in a desired position under the patient's skin. With smaller sensor members, dislodging of the sensor member, micromovements, and potential unintended withdrawal of the entire sensor member from the patient's skin all become more prominent issues. Therefore, in addition to the structure of the sensor member described above, additional safeguards may also be implemented. One such additional consideration according to embodiments of the invention is the insertion angle at which the sensor is implanted.

FIG. 5 provides a side cross-sectional view of a schematic sensor member 1 of an analyte monitor that is implanted in skin 1000 according to an embodiment of the invention, while FIG. 6 shows a cutaway view of a portion of a patient's skin with a schematic depiction of the sensor member 1 implanted therein according to an embodiment of the invention. As best seen in FIG. 6, the skin 1000 of a human includes various layers, such as an epidermis 1100 that is closest to the surface of the skin, an intermediate dermis 1200, and a deeper hypodermis 1300. Typical sensors on the market (not shown) are larger and therefore generally extend more deeply into the skin, with sensing regions that are typically positioned in the hypodermis 1300. As such, most conventional sensor members need to both be long enough to extend into the hypodermis 1300, as well as sturdy enough to be held at that depth, thereby generally necessitating a thicker overall sensor member as well. Consequently, particularly in the case of continuous glucose monitors that may be worn for longer durations such as multiple days or weeks, these longer sensors may be more abrasive, and/or cause more trauma or discomfort to the patient, both during insertion or implantation, as well as during the life of the sensor. Furthermore, the hypodermis 1300 of the skin 1000 is generally more fatty than the epidermis 1100 and the dermis 1200, and so when the sensing region is positioned in the hypodermis 1300, the increased levels of fat in the hypodermis 1300 may delay glucose transport, and cause a time lag associated with a useable signal from the sensor member. In some cases, the time lag caused by the increased fat levels in the hypodermis 1300 compared to the dermis 1200 or epidermis 1100 before a useable signal can be obtained by the sensor member may be 15 minutes or more, which may cause a significant delay in data collection and analysis by the monitor, as well as potential inaccuracies if an inaccurate signal is used or incorporated into later data analyses.

In contrast, as schematically shown in FIG. 6, sensor members 1 according to embodiments of the invention may only extend more shallowly into the shallow regions of the dermis 1200, with sensing regions in either the shallow regions of the dermis 1200, the epidermis 1100, or both, rather than requiring the sensing regions to extend more deeply into the hypodermis 1300. Positioning the sensing regions of the sensor member 1 in the shallow regions of the dermis 1200 or the epidermis 1100 may have many advantages. For example, the smaller size of the sensor member 1 according to embodiments of the invention, both in terms of length and thickness, may reduce trauma during insertion and/or discomfort during the life of the sensor/monitor. The reduction in size of the sensor member 1 compared to traditional sensor members, or more generally, the reduced depth to which the sensor member 1 extends under the skin, will also allow sensing in less fatty layers of the skin, consequently resulting in faster glucose transport, more accurate signal readings by the sensor member, and a shorter delay or time lag associated with being able to retrieve useable signals from the sensor member 1. Generally speaking, sensor members 1 according to embodiments of the invention may extend to approximately ⅓ of the depth of traditional sensor members, but the sensor member in other embodiments may be positioned shallower or deeper compared with traditional sensor members, without departing from the spirit or scope of the invention, so long as the sensing regions are not intended to be positioned deeper than the shallow regions of the dermis 1200. Targeting the shallower regions of the dermis 1200 furthermore allows for some tolerance or leeway, where for example, if a sensor member 1 is implanted at a slightly more vertical angle than intended, the sensing regions of the sensor member 1 should still be positioned in the dermis 1200 rather than deeper in the hypodermis 1300, and still benefit from both reduced discomfort and less glucose transport delays due to fat in the tissue.

Referring now to FIG. 5, when the sensor member 1 is implanted at a shallower depth, retention issues that are not present for deeper extending sensor members may arise. Movements by the patient, for example, manipulation of the skin or underlying muscles or other tissue, may cause the implanted portion of the sensor member 1 to move, and when the sensor member does not extend as deeply into the skin, such movements may cause the implanted portion of the sensor member 1 to partially withdraw, or may potentially even completely pull out of the skin. This issue may be further exacerbated in situations where the sensor member 1 is implanted into the skin at an angle substantially perpendicular to the surface of the skin, where in such cases, the length of the sensor member 1 implanted into the skin is the same as the depth of the sensor member, so that a movement that dislodges the sensor member 1 or its corresponding monitor by about that length/depth may potentially cause an unwanted withdrawal or pulling out of the sensor member 1 from the skin, which would disrupt the monitoring of the patient.

As such, as shown in FIG. 5, according to embodiments of the invention, the sensor member 1 can be implanted under the skin at an acute entry angle α relative to a surface plane of the skin 1000 instead. By implanting sensor member 1 at an angle α, the length of the sensor that is actually implanted into the skin can be increased. For example, as shown in FIG. 5, if the entry angle α is 30° relative to the surface of the skin 1000 and the sensor member 1 is implanted to a depth D, the actual length L of the portion of the sensor member 1 that is implanted will be two times the depth D. Just by way of example, if the entry angle α is 45° relative to the surface of the skin 1000, the actual length L of the portion of the sensor member 1 that is implanted will be about 1.4 times the depth D. Therefore, by varying the angle of implantation, a larger portion of the sensor member 1 can be implanted under the skin 1000 without the sensor member reaching an undesirably large depth, e.g., without the sensor member 1 entering the hypodermis. And by way of a larger length of sensor member 1 being implanted under the skin 1000, retention of the sensor member 1 under the skin 1000 can be improved, so that it will be more difficult to dislodge the sensor member 1 from withdrawing and pulling out of the skin 1000. Furthermore, an angled insertion according to embodiments of the invention will allow for more consistent placement of the sensor member 1 in the correct tissue space, e.g., the epidermis or the shallow dermis, and will provide more tolerance or leeway to do so.

Placement of the sensing regions of the sensor members according to embodiments of the invention in the epidermis or shallow dermis will both provide for a more homogeneous environment in which the sensing regions will reside, as well as cause less trauma to the surrounding tissue, thereby leading to a more favorable wound healing response, and allowing for more natural circulation of interstitial fluid, which should further result in both a more homogeneous environment around the sensing regions, as well as a quicker settling time after sensor delivery for reaching such conditions.

In the presence of an amorphously structured matrix like human skin, when using smaller scale sensor members according to embodiments of the invention, charge flux uniformity across the electrode surface and tissue displacement may be optimized in these small area electrode systems due to the reduced scale of physical interactions with the sensor member's immediate surroundings in the skin. These benefits, coupled with the mitigation of resistive contributions to the system, all contribute to improved performance in accuracy and stability of such electrochemical biosensors with sensing regions that have reduced size compared to competitor sensor members. With respect to accuracy, improvements in mean absolute relative difference (MARD) have been observed, where the average difference between readings from analyte monitors according to embodiments of the invention and expected reference measurements is noticeably lower compared to prior existing designs. With respect to stability, noise levels associated with readings from analyte monitors according to embodiments of the invention have also been observed to be lower compared to prior existing designs, described in greater detail below.

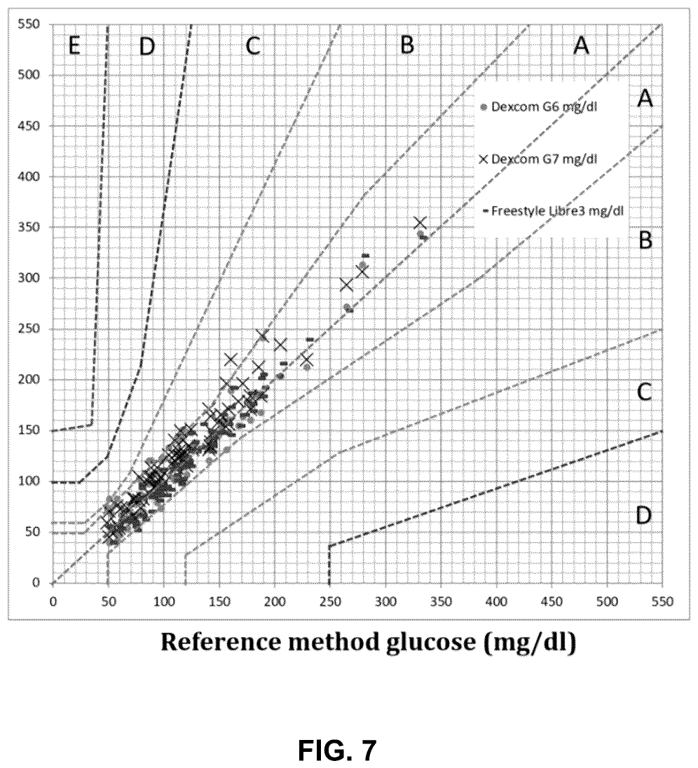

FIG. 7 shows a consensus error grid that charts typical performance characteristics for various competitor devices that are currently on the market, based on an article from DiabetTech, “Battle Royale—Freestyle Libre 3 and Dexcom G7 face off—The Results” (https://www.diabettech.com/cgm/battle-royale-freestyle-libre-3-and-dexcom-g7-face-off-the-results/). The study tracked performance of DexCom G6, DexCom G7, and FreeStyle Libre 3, three of the more popular glucose monitors currently available. When measuring glucose and comparing with expected reference measurements, the results for each monitor, along with the overall results, returned a relatively large dispersion away from the expected reference measurements, where readings that match the expected reference measurements would fall on the central diagonal line that extends through the center of zone A. Typically, when viewing a consensus error grid, results within zone A are generally considered accurate, as there should be no potential impact on clinical decision making if the results fall within zone A. However, results that fall within zone B represent quantifiable errors that may result in a change in the proposed clinical action or decision, and results that fall within zone C represent quantifiable errors that may result in both a change in the proposed clinical action or decision, as well as altered or different clinical outcomes. As such, the most desirable results are (1) as close to the expected reference measurement line as possible, and (2) at least not outside of zone A. As can be seen, particularly for glucose measurements below 150 mg/dL, a significant number of measurements fell outside of zone A into zone B, and at least one measurement made by the DexCom G6 at 50 mg/dL fell outside of zone B into zone C.

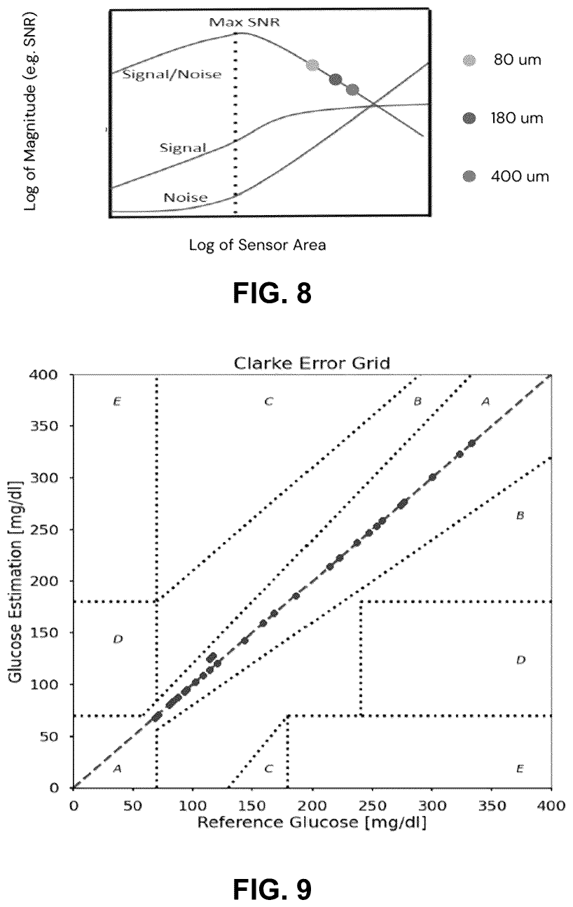

As discussed above, one of the main contributors to inaccurate signals and increased noise is caused by the large size of the sensing regions on a sensor member, due to various different factors. FIG. 8 is a graph that shows respective relationships between sensor area and signal strength, noise levels, and signal-to-noise ratio, taken from an analysis in Analytical Chemistry, Vol. 50, No. 13, November 1984, with additional plots added based on testing using sensor members with different sized sensing regions. As can be seen in FIG. 8, while a slope/magnitude of the signal starts to reduce and plateau as sensor area increases, the noise levels continue to rise consistently with increased sensing region sizes. Therefore, as the size of sensing regions increases, the signal-to-noise ratio begins to reduce significantly past a certain point. When testing sensor members with different sized sensing regions, it was observed that sensors with 180 μm sensing regions had a higher signal-to-noise ratio than sensors with 400 μm sensing regions, and that sensors with 80 μm sensing regions had an even higher signal-to-noise ratio than sensors with 180 μm sensing regions. Meanwhile, while not pictured, it further follows that sensors with 40 μm sensing regions according to embodiments of the invention have even higher signal-to-noise ratios and perform even better than sensors with 80 μm sensing regions. Of course, at a certain point, we once again run into manufacturing limitations where it may no longer be possible to make smaller sensing regions with the current technology, and further, sensors with sensing regions that are too small may in any case see reduced performance with respect to signal-to-noise ratio as well, due to reduced signal magnitudes, based on the graph in FIG. 8.

In view of the above, sensors with sensing regions having reduced length/height, e.g., on the order of about 40 μm to 80 μm according to embodiments of the invention were tested for accuracy, and FIG. 9 is the resulting Clarke Error Grid for various measurements that were taken at different glucose concentrations, compared to the reference glucose measurements. Clark Error Grids chart performance of analyte monitors compared to reference glucose measurements similarly to consensus error grids, but consider slightly different error zones or regions when analyzing the test results. Clark Error Grids have been considered a standard accuracy metric for continuous glucose monitors specifically. As can be seen in FIG. 9, based on the results in the Clarke Error Grid, glucose estimation by analyte monitors according to embodiments of the invention very closely tracked the reference glucose measurements, as nearly all plots fell on or close to the central diagonal line which represents results that match the reference glucose measurements. While two readings showed a slightly positive bias at about 120 mg/dL glucose, the measurements were nevertheless safely within region A, which reflects a minimal deviation from the expected glucose measurements (e.g., a deviation of less than 20% from expected results) and generally no potential impact on clinical decision making.

Therefore, as can be seen from testing results, sensor members with reduced sensing regions according to embodiments of the invention yielded much more accurate glucose readings, compared to other commercially available glucose sensors. Similarly as previously discussed, typical glucose sensors are sensitive to various sources of biological and environmental noise, which may be reduced or eliminated in the case of sensor members with smaller sensing regions according to embodiments of the invention, which is reflected in the Clarke Error Grid in FIG. 9 where such sources of noise were not in evidence or observed, or at least affected the sensor readings much less prominently. As such, sensor members with smaller sensing regions according to embodiments of the invention have demonstrated both increased accuracy and reduced noise, among other benefits such as general reduced discomfort for the patient.

Other embodiments may also be envisioned without departing from the spirit or scope of the invention. Various modifications can also be incorporated while retaining the inventive concepts of the present application.

In addition to the embodiments that have already been described above, it is also possible to combine embodiments, e.g., different features from the various described embodiments, to provide even more different variations of sensor members and/or associated analyte monitors, without departing from the spirit or scope of the invention. In addition, the inventions should not be limited to the structures and/or shapes described in the embodiments above.

While the subject matter of the present disclosure has been described in connection with certain embodiments, it is to be understood that the subject matter of the present disclosure is not limited to the disclosed embodiments, but, on the contrary, the present disclosure is intended to cover various modifications and equivalent arrangements included within the spirit and scope of the appended claims, and equivalents thereof.

1. A monitor for determining analyte concentrations in vivo, comprising:
a housing configured to adhere to the skin of a subject;
a sensor body configured to extend from the housing into a patient's skin, the sensor body being elongate and extending along a longitudinal axis in a length direction, and further having a width and a thickness each measured perpendicular to the length; and
an analyte sensing region formed on the sensor body, wherein the analyte sensing region has opposite ends in the length direction, and wherein a maximum length of the analyte sensing region measured between the opposite ends is less than 80 μm.

2. The monitor of claim 1, wherein the maximum length of the analyte sensing region measured between the opposite ends is 40 μm or less.

3. The monitor of claim 1, wherein the monitor is a glucose monitor configured to determine glucose levels of the patient in vivo.

4. The monitor of claim 1, wherein the analyte sensing region is positioned near a free end of the sensor body.

5. The monitor of claim 1, wherein a width of the sensor body approximate the analyte sensing region is greater than a width of another portion of the sensor body away from the analyte sensing region.

6. The monitor of claim 1, wherein when the sensor body extends into the patient's skin, the analyte sensing region is configured to be held in the dermis of the skin of the patient.

7. The monitor of claim 1, wherein the analyte sensing region comprises a plurality of electrodes.

8. The monitor of claim 7, wherein the plurality of electrodes comprises a working electrode.

9. The monitor of claim 8, wherein the plurality of electrodes further comprises at least one of a counter electrode or a reference electrode.

10. The monitor of claim 1, wherein when the sensor body extends into the patient's skin, a free end of the sensor body is configured to be held in the dermis of the skin of the patient.

11. The monitor of claim 1, wherein when the sensor body extends into the patient's skin, the sensor body extends at an acute angle to a surface of the patient's skin.

12. The monitor of claim 1, wherein the housing comprises a base having a surface configured to adhere to the skin of the subject and a separate transmitter configured to be assembled to the base after the sensor body is implanted into the patient's skin.

13. A sensor member for a monitor that is configured to determine analyte concentrations in vivo, wherein the sensor member comprises:
a sensor body configured to extend into a patient's skin, the sensor body being elongate and extending along a longitudinal axis in a length direction; and
an analyte sensing region that is formed on the sensor body by utilizing at least one microfabrication technique, wherein the analyte sensing region has opposite ends in the length direction, and wherein a maximum length of the analyte sensing region measured between the opposite ends is less than 80 μm.

14. The sensor member of claim 13, wherein the maximum length of the analyte sensing region measured between the opposite ends is 40 μm or less.

15. The sensor member of claim 13, wherein the monitor is a glucose monitor configured to determine glucose levels of the patient in vivo.

16. The sensor member of claim 13, wherein the sensor body has a length such that when the sensor body extends into the patient's skin, a free end of the sensor body is configured to be held in the dermis of the skin of the patient.

17. The sensor member of claim 13, wherein the at least one microfabrication technique comprises photo lithography.

18. The sensor member of claim 13, wherein the at least one microfabrication technique comprises ultraviolet (UV) etching.

19. The sensor member of claim 13, wherein the at least one microfabrication technique comprises electrochemical plating.

20. The sensor member of claim 13, wherein the sensor body is formed on a wafer, and wherein the at least one microfabrication technique comprises layering functional chemistry on the wafer by spinning as material substrates are deposited as liquid.
